(19) United States
(12) Patent Application Publication
(10) Pub. No.: US 2023/0115039 A1
(43) Pub. Date: Apr. 13, 2023

(54) MACHINE-LEARNING TECHNIQUES FOR PREDICTING SURFACE-PRESENTING PEPTIDES

(71) Applicant: Personalis, Inc., Menlo Park, CA (US)

(72) Inventors: Charles Wilbur ABBOTT, III, Menlo Park, CA (US); Sean Michael BOYLE, Menlo Park, CA (US); Rachel Marty PYKE, Menlo Park, CA (US); Eric LEVY, Menlo Park, CA (US); Dattatreya MELLACHERUVU, Menlo Park, CA (US); Rena MCCLORY, Menlo Park, CA (US); Richard CHEN, Menlo Park, CA (US); Robert POWER, Menlo Park, CA (US); Gabor BARTHA, Menlo Park, CA (US); Jason HARRIS, Menlo Park, CA (US); Pamela MILANI, Menlo Park, CA (US); Prateek TANDON, Menlo Park, CA (US); Paul MCNITT, Menlo Park, CA (US); Massimo MORRA, Menlo Park, CA (US); Sejal DESAI, Menlo Park, CA (US); Juan-Sebastian SALVIDAR, Menlo Park, CA (US); Michael CLARK, Menlo Park, CA (US); Christian HAUDENSCHILD, Menlo Park, CA (US); John WEST, Menlo Park, CA (US); Nick PHILLIPS, Menlo Park, CA (US); Simo V. ZHANG, Menlo Park, CA (US)

(73) Assignee: Personalis, Inc., Menlo Park, CA (US)

(21) Appl. No.: 18/065,410

(22) Filed: Dec. 13, 2022

Related U.S. Application Data
(63) Continuation of application No. PCT/US2021/037902, filed on Jun. 17, 2021.
(60) Provisional application No. 63/040,943, filed on Jun. 18, 2020.
(60) Provisional application No. 63/111,007, filed on Nov. 7, 2020.

(51) Int. Cl. G16B 40/20; G16B 25/10; G06N 20/20

(52) U.S. Cl. CPC G16B 40/20; G06N 20/20; G16B 25/10

(57) ABSTRACT

The disclosure provides methods for predicting surface-presenting peptides using binding and surface-presentation characteristics. The method can include accessing a trained machine-learning model that is configured to generate an output that indicates an extent to which the one or more expression levels and the one or more peptide-presentation metrics are related in accordance with a population-level relationship between expression and presentation. For each peptide of the set of peptides for a tissue sample, a score can be determined using the machine-learning model and genomic and transcriptomic data corresponding to the peptide. The score is predictive of whether a corresponding peptide is a surface-presenting peptide that binds to an MHC molecule and is presented on a cell surface.

### CROSS-REFERENCES TO RELATED APPLICATIONS

The present application is a continuation of International Application No. PCT/US2021/037902, filed on Jun. 17, 2021, and claims priority to U.S. Provisional Application No. 63/040,943, entitled “Composite Biomarkers for Immunotherapy for Cancer” filed Jun. 18, 2020, and U.S. Provisional Application No. 63/111,007, entitled “Machine-Learning Techniques For Predicting Surface-Presenting Peptides” filed Nov. 7, 2020, the entire contents of which are herein incorporated by reference in their entirety for all purposes.

### BACKGROUND OF THE INVENTION

Cancers include mutations, which may be somatic and tumor-specific. The immune system detects these cancer-based mutations by identifying peptides that are derived from these mutations. The peptides can be identified by the immune system when they bind proteins encoded by a major histocompatibility complex (MHC) gene and are presented on a surface of a cell. For example, a peptide corresponding to a mutated gene can bind to a specific MHC molecule (e.g., human leukocyte antigen (HLA) protein) and be presented on the cell surface. Predicting peptides expressed on a tumor cell surface can inform development of precision cancer therapeutics and diagnostics. For example, genomic variants corresponding to these peptides can be identified to analyze complex systems’ responses and resistance to certain cancer immunotherapies. As another example, the peptides presented on the tumor cell surface can be analyzed to create personalized immuno-oncology (I-O) therapies and/or neoantigen cancer vaccines.

Techniques for predicting such peptides expressed on tumor cell surface, also known as “neoantigens”, require in-depth analysis of many technical factors, including, but not limited to, the quality of peptide sequencing data, availability of paired tumor and normal samples, HLA-typing, and identification of other peptide characteristics. For example, a neoantigen can be identified based on a prediction of a peptide that will bind to an MHC molecule and be presented on a cell surface. To identify neoantigens, determining peptides encoded by somatic variants and identifying HLA molecules that bind the peptides are only the initial steps in a very complex process. This is because each peptide identified from the sequence data may or may not be: processed by the proteasome; transported for MHC binding; presented on a tumor cell surface; and ultimately recognized by the immune system. Because of this complex process, many peptides that bind to an HLA molecule (for example) may not be expressed on the cell surface.

Further, one or more binding motifs of the MHC molecules can be identified to determine whether a given peptide will bind to the MHC molecule. While binding motifs for some MHC molecules (e.g., HLA-A molecule) are known, there are many MHC molecules for which binding motifs are yet to be identified. For example, binding motifs of MHC Class II molecules are relatively unknown due to limited availability of experimental data. Without this information, it would be difficult to determine whether a peptide will bind to a corresponding MHC molecule. Conventional techniques have attempted to address this issue by training machine-learning models using known MHC binding motifs to predict whether a peptide will bind to one of the various types of MHC molecules. However, even when such peptides are identified, some of them may not be presented on a cell surface. In other words, conventional techniques may identify MHC-binding peptides, but only a small fraction of them can be successfully presented on a cell surface. Since an immune system response is triggered when MHC-binding peptides are presented on the cell surface, identifying MHC-binding peptides alone cannot provide all the details on how the immune system responds to tumor cells, foreign protein, etc.

Thus, conventional techniques for predicting MHC-binding peptides do not address whether the peptides are actually presented and expressed on a cell surface. Conventional techniques also fall short of identifying peptide characteristics that are indicative of a given peptide being presented on the cell surface. Accordingly, there is a need for accurately predicting peptides that bind to their corresponding MHC molecules and are presented on a cell surface.

### BRIEF SUMMARY OF THE INVENTION

In some embodiments, a method of predicting surface-presenting peptides is provided. The method can include accessing a trained machine-learning model, which was trained using a training data set that included, for each peptide of a plurality of peptides identified by the training data set, protein characteristics of a major histocompatibility complex (MHC) molecule that binds and presents the peptide, one or more expression levels representing an expression level of a gene encoding the peptide, and one or more peptide-presentation metrics representing a quantity of peptides detected as having been presented by the MHC molecule. The machine-learning model can be configured to generate an output that indicates an extent to which the one or more expression levels and the one or more peptide-presentation metrics are related in accordance with a population-level relationship between expression and presentation.

The method can also include accessing genomic and transcriptomic data corresponding to a biological sample of a subject. The genomic and transcriptomic data can identify one or more MHC molecules from the biological sample and include, for each peptide of a set of peptides identified from the cell line or tissue samples, one or more values representing the peptide. The one or more values can be determined based on processing of the tissue sample. The method can also include determining, for each peptide of the set of peptides, a score using the machine-learning model, the one or more MHC molecules identified from the biological samples, and the one or more values representing the peptide. The method can include generating a result based on the score and outputting the result.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates a schematic diagram of a peptide binding to an MHC molecule and presented on a cell surface.

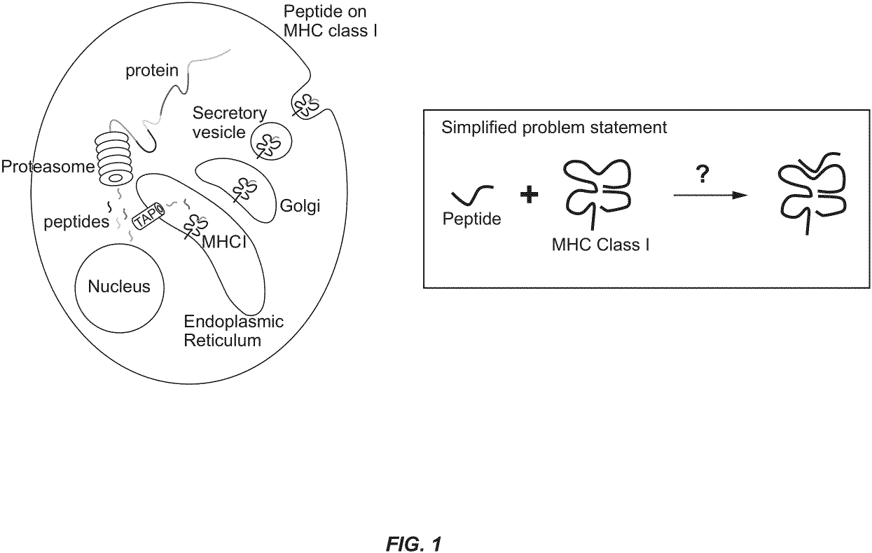

FIG. 2 illustrates a schematic diagram that shows peptides that can be presented on a cell surface in response to gene therapies.

FIG. 3 illustrates a schematic diagram of identifying mono-allelic immunopeptidomics data that can be used for training a machine-learning model, in accordance with some embodiments.

FIG. 4 shows allelic diversity data corresponding to MHC-binding peptides, according to some embodiments.

FIG. 5 shows source diversity data identified from tissue and cell line samples for training a machine-learning model for predicting surface-presenting peptides, in accordance with some embodiments.

FIG. 6 shows a plot of comparison data between expected peptide counts based on gene expression levels and actual observed peptide counts, according to some embodiments.

FIG. 7 shows a process for determining a gene propensity score used for training a machine-learning model, according to some embodiments.

FIG. 8 shows a plot of comparison data between expected peptide counts for one or more regions within a gene and actual observed peptide count for the regions, according to some embodiments.

FIG. 9 shows a process for determining a hotspot score used for training a machine-learning model, according to some embodiments.

FIG. 10 shows an example of features used by a binding model and a presentation model, according to some embodiments.

FIG. 11 illustrates an exemplary model architecture for training a machine-learning model for predicting surface-presenting peptides, according to some embodiments.

FIG. 12 shows performance levels of trained binding models and trained presentation models based on 10% held-out data and measured in terms of positive predictive value, according to some embodiments.

FIG. 13 shows a comparison of performance levels of a trained machine-learning model compared to conventional techniques for predicting surface-presenting peptides.

FIG. 14 shows a comparison of performance levels of a trained presentation model across various alleles, compared to conventional techniques for predicting surface-presenting peptides.

FIG. 15 shows results from a leave-one out analysis of a trained presentation model, according to some embodiments.

FIG. 16 shows a graph depicting precision and recall values for evaluating a trained machine-learning model, according to some embodiments.

FIG. 17 shows a box plot that indicates performance levels of a trained machine-learning model across different tissue samples, according to some embodiments.

FIG. 18 shows a graph that compares performance levels of trained machine learning models and other conventional techniques, according to some embodiments.

FIG. 19 includes a flowchart illustrating an example of a method of predicting surface-presenting peptides, according to certain embodiments.

FIG. 20 illustrates an example of a computer system for implementing some of the embodiments disclosed herein.

### DETAILED DESCRIPTION OF THE INVENTION

### I. Overview

To address at least the above deficiencies of conventional systems, the present techniques can be used to predict surface-presenting peptides. As used herein, a “surface-presenting peptide” can refer to a peptide that binds to an MHC molecule (e.g., an HLA-A protein) and is presented on a corresponding cell surface. One or more somatic variants can be identified by sequencing DNA from normal and tumor samples. A somatic variant includes one or more gene mutations present in the tumor sample and also in the normal sample. The somatic variant of the tumor sample can be processed using a trained machine-learning model to predict whether a peptide encoded by the somatic variant will bind to an MHC molecule (e.g., MHC Class 1) and be presented on a cell surface. The machine-learning model can include a binding model that predicts whether a peptide encoded by the somatic variant will bind to an MHC molecule. In some embodiments, the machine-learning model includes a presentation model that predicts whether the peptide encoded by the somatic variant will be expressed on a cell surface.

The machine-learning model can be trained using a training data set derived from: (i) genetically engineered mono-allelic cell lines; and (ii) multi-allelic data from tissue samples of other subjects. In some instances, the machine-learning model was trained using binding array data (e.g., IEDB data). The training data set can include, for each peptide identified by the training data set, one or more expression levels representing an expression level of a gene encoding the peptide and one or more peptide-presentation metrics representing a quantity of peptides detected as having been presented by the MHC molecule. The training data set can include immunopeptidomics data of peptides generated from a plurality of genetically engineered cell lines (e.g., K562 cells) that express a single allele of interest (e.g., HLA-A). In particular, MHC-peptide complexes in these cell lines may be immunoprecipitated using W6/32 antibody, followed by peptide elution and peptide sequencing using tandem mass spectrometry. The training data set corresponding to multi-allelic data from other tissue samples can be obtained using curated public data.

The prediction of surface-presenting peptides can be performed in a manner that biases the selection towards peptides associated with scores predicting presentation to be more probable relative to a probability expected by a population-level relationship between expression and presentation of the peptides. Additionally or alternatively, a prediction of surface-presenting peptides is performed in a manner that biases the selection towards peptides associated with a region in a space, the region being associated with outlier peptides in the training data set for which expression levels and peptide-presentation metrics were related in a manner that departed from the population-level relationship.

Accordingly, embodiments of the present disclosure provide a technical advantage over conventional systems by accurately predicting peptides that bind to their corresponding MHC molecules and are presented on a cell surface. As noted above, binding and expression of peptides on a tumor cell surface can be predictive of how immune systems will respond to neoantigens and/or certain cancer immunotherapies. Thus, an accurate prediction of surface-presenting peptides facilitates selection or development of immunotherapies that would be most effective for a given subject. Further, based on the model evaluations, the embodiments demonstrate a significantly higher positive predictive value compared to conventional techniques such as NetMHCPan 4.0. As such, the high sensitivity and specificity of the embodiments enable accurate identification of MHC-binding peptides that are presented on a cell surface, thereby facilitating applications to the development of personalized immunotherapies and biomarker discovery.

The following examples are provided to introduce certain embodiments. In the following description, for the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples. The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as an “example” is not necessarily to be construed as preferred or advantageous over other embodiments or designs.

### II. Surface Presentation of Peptides

### 1. Neoantigens in Tumor Samples

Neoantigens can be found in tumor samples, in which the neoantigens indicate one or more peptides that are presented on the tumor-cell surface thereby triggering an immune-system response. The immune system can be conditioned to seek pathogens including cancer and thus has the capacity to cure cancer. The immune system can distinguish self from non-self antigens. Because tumors are caused by genetic mutations (e.g., somatic variants), peptides corresponding to these genetic mutations and expressed on a cell surface can be considered as neoantigens. Because these peptides are considered “new” to the immune system, the immune system can ideally recognize tumor cells based on detecting the neoantigens presented on a tumor-cell surface and eliminate the tumor cells. As explained above, a tumor sample can be analyzed to identify sequence data and the sequence data can be compared with those from a normal sample to identify somatic variants. The somatic variants can be further analyzed to determine which subset of the variants will be manifested as peptides. Neoantigens can be predicted by identifying peptides that bind to an MHC molecule and are presented on the cell surface. Thus, a peptide’s ability to be presented on a cell surface can be a key component for developing immunotherapies against cancer.

### 2. Peptides in Response to Treatments for Certain Autoimmune Diseases

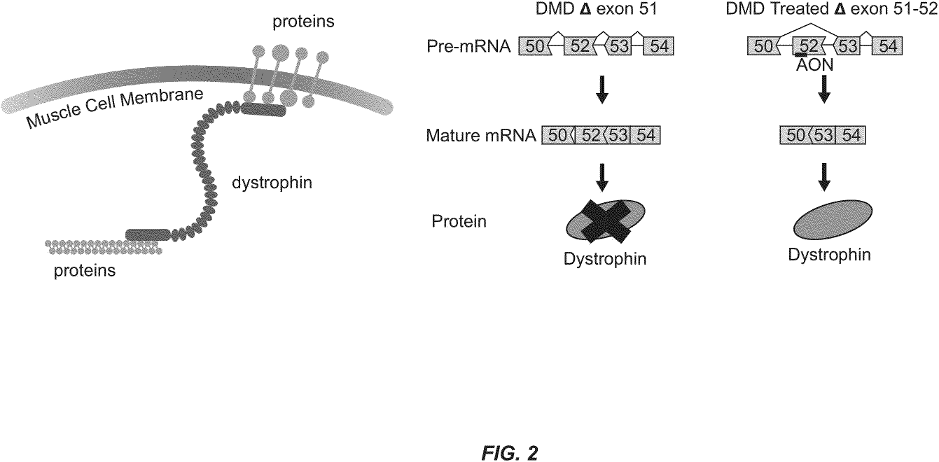

Surface-presented peptides can be identified in the context of autoimmune diseases, in which the peptides are encoded based on genetic alterations resulting from particular immunotherapies. FIG. 2 illustrates a schematic diagram that shows surface-presenting peptides in response to gene therapies. In FIG. 2, a mutation in the dystrophin gene is shown, which typically results in muscular dystrophy which is debilitating. The dystrophin gene encodes dystrophin protein molecules that serve as a cushion as a shock absorber in muscle cells. Absence of the fully functional dystrophin protein can lead to degeneration of the muscle. Typically, muscular dystrophy can be treated exome skipping therapy, in which exomes causing the dystrophin gene mutation can be skipped (e.g., exon 52) to generate a semi-functional dystrophin protein for the subject. Although the exome skipping therapy can be effective, it may trigger generation of new types of peptides due to exomes being intentionally skipped through genetic alteration. The new peptides may end up binding to the MHC molecule and presented on the cell surface, thereby triggering potentially destructive immune system response.

### III. Example Training Datasets

The machine-learning models for predicting surface-presenting peptides can be trained using a supervised training algorithm. The machine-learning models can be trained using a training data set. The training data set for training the machine-learning models can include sequence data from various sources: (i) peptides identified as binding to HLA molecules based on in vitro experiments; (ii) peptides identified by performing mass spectrometry from tumor samples; (iii) HLA alleles; and (iv) non-tumor samples. However, some training sequence data may be inaccurate for training the machine-learning models. For example, training sequence data generated from tissue samples would require a difficult process of mapping peptides to one of several types of HLA proteins (e.g., HLA-A, HLA-B) that are being simultaneously expressed on the cell surface. In another example, sequence data generated using in vitro methods might not model surface presentation. Embodiments of the present disclosure to systematically resolve inconsistencies in the training data set is used to train the machine-learning model to predict, from somatic variants called from sequence data, a peptide that is likely to be “shuttled” to the cell surface.

Additionally or alternatively, the training data set may further include data corresponding to somatic variants, in which each somatic variant is labeled to indicate whether a peptide encoded by the somatic variant will bind to an MHC molecule (e.g., an HLA-A protein) and presented on a cell surface. The training data set can also include one or more features derived from the somatic variants (e.g., peptide sequence, peptide length, expression of peptide in the tumor sample).

To prepare the training data set, a tumor sample and a corresponding normal, control sample can be sequenced to generate a tumor-normal pair sequence data. The tumor-normal pair sequence data are compared to identify somatic variants, including altered genes that include single-nucleotide variants (SNV), indels, and/or copy number variations. In some instances, a machine-learning model is used to process the tumor-normal pair sequence data to identify the somatic variants in the tumor sample.

### 1. Training Data Sources

### a) Mono-Allelic Immunopeptidomics Data

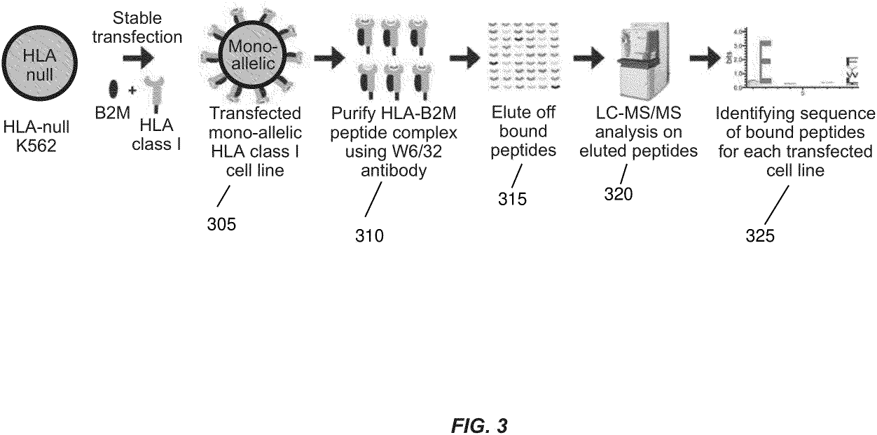

In some instances, at least some of the training data correspond to peptides identified from genetically engineered mono-allelic cell lines. FIG. 3 illustrates a schematic diagram of identifying mono-allelic immunopeptidomics data that can be used for training a machine-learning model, in accordance with some embodiments. As shown in FIG. 3, genetically engineered mono-allelic K562 cell lines can be created then transfected with a particular HLA molecule of interest (e.g., HLA-B) (step 305). As explained above, HLA complex is a group of related proteins that are encoded by the MHC gene complex in humans. These cell-surface proteins are responsible for the regulation of the immune system. From the cell-lines, HLA-binding peptides can be identified by immunoprecipitating HLA-peptide complexes using W6/32 antibody (step 310), applying peptide elution (step 315), and performing peptide sequencing on the eluted peptides using mass spectrometry (e.g., a liquid chromatography-mass spectrometry mass spectrometry) (step 320). The HLA-binding peptides can thus be identified for that particular HLA molecule of interest (step 325).

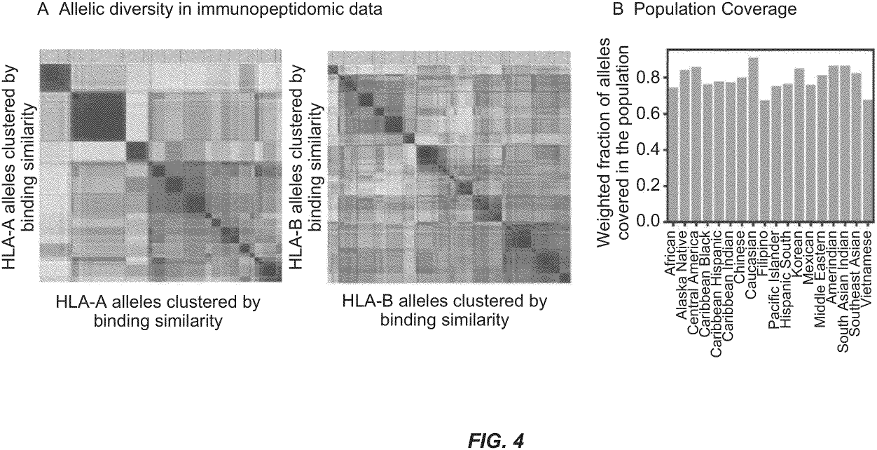

Mono-allelic immunopeptidomics data identifying various characteristics of the HLA-binding peptides can be identified and included as part of the training data set. Examples of training data from mono-allelic immunopeptidomics data can include, for a given HLA-binding peptide, a type of peptide, length of the peptide, amino-acid sequence of the peptide, an HLA allele that binds the peptide, a number of transcripts that correspond to the peptide, and expression of a gene region that encodes the peptide. In order to optimize the performance of the machine-learning model, training data was generated to be representative of HLA genotypes in the general population. For example, FIG. 4 shows allelic diversity data corresponding to HLA-binding peptides, according to some embodiments. To determine the allelic diversity data, the identified peptides can be clustered based on their similarities with respect to peptide sequences corresponding to all known alleles of the HLA molecule of interest (e.g., HLA alleles identified from IMGT database). The identified peptides can thus be clustered based on their respective binding pocket similarities. In some instances, the identified peptides can be clustered using a BLOSUM similarity matrix. Based on these clusters, one or more alleles that encode the HLA-binding peptides can also be identified. In some instances, the peptide clusters are visualized on a heatmap. For example, FIG. 4 shows a first heat map that identifies allelic diversity for HLA-A molecule and a second heat map that identifies allelic diversity for HLA-B molecule. Additionally or alternatively, the training data set can be enhanced using training data corresponding to allele frequency data of alleles encoding HLA-binding proteins, in which the allele frequency data is categorized into different parts of the world population.

The training data corresponding to the HLA-binding peptides can thus facilitate training of machine-learning model by using mono-allelic immunopeptidomics data that express one particular type of HLA at a time. Further, allelic diversity in the mono-allelic immunopeptidomics training data enables the machine-learning model to predict surface-presenting peptides derived from various alleles that may be absent from the training data.

### b) Multi-Allelic Immunopeptidomics Data

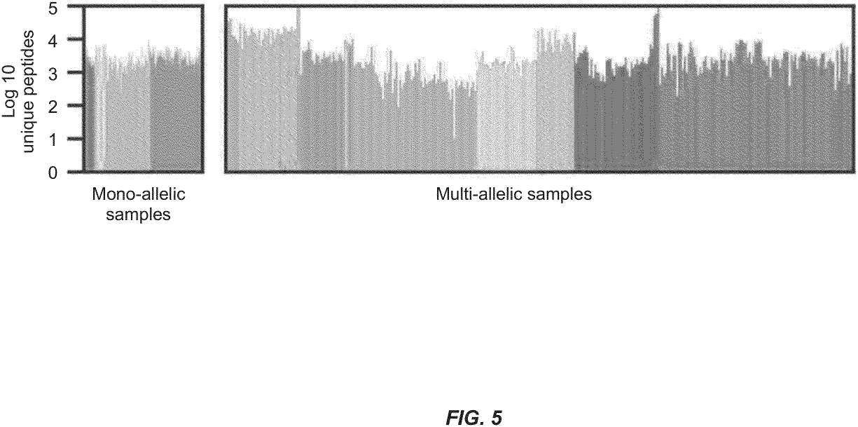

In some instances, at least some of the training data correspond to peptides identified from sequencing tissue samples of other subjects. Cell lines of different tissue samples or tissue samples of subjects can be sequenced to identify a plurality of peptides that bind to different types of HLA molecules (e.g., HLA-A, HLA-B, HLA-C). In some instances, the cell lines and tissue samples are processed using mass spectrometry. Multi-allelic immunopeptidomics data derived from the identified plurality of peptides can be used as part of the training data. The multi-allelic immunopeptidomics data may include various characteristics corresponding to the identified peptides, including peptide length and allelic diversity. FIG. 5 shows source diversity data identified from tissue samples of subjects for training a machine-learning model for predicting surface-presenting peptides, in accordance with some embodiments. In FIG. 5, quantity for each peptide type are shown for each of the mono- and multi-allelic samples. Additionally or alternatively, the multi-allelic data can be obtained from a public data source.

The multi-allelic immunopeptidomics data generated from diverse tissues and cell lines can be integrated into the training data set to improve the performance of the trained machine-learning model. In particular, training the machine-learning model with multi-allelic immunopeptidomics data may reduce overfitting and/or underfitting. For example, both mono-and multi-allelic immunopeptidomics data from several publicly available data sources can be added into the training data set. The mono-allelic immunopeptidomics data from genetically engineered cell lines and mono- and multi-allelic immunopeptidomics data from tissue samples can all be combined into the training data set to expand its scale (e.g., a greater quantity of unique peptide counts).

### 2. Additional Enhancing Features

As explained above, the immunopeptidomics data from the training data set identify various characteristics of an HLA-binding peptide, including peptide sequence, peptide length, a binding pocket sequence, left flanking region, and right flanking region. In some instances, the training data set also includes antigen presentation features such as expression level of peptides measured in terms of DPM. In addition to the above, two additional features can be generated from the immunopeptidomics data, which can be used to enhance the training data set.

### a) Comparison Data Between Expected Peptide Counts Based on Gene Expression Levels and Actual Observed Peptide Count

A first feature generated from the immunopeptidomics data can include comparison data between expected peptide counts based on gene expression levels and actual observed peptide count. By including the training data set with the first feature, the trained machine-learning model trained from the above training data can improve prediction of surface-presenting peptides such that the prediction is biased towards peptides associated with scores predicting presentation to be more probable relative to a probability expected by a population-level relationship between expression and presentation of the peptides. In addition, the trained machine-learning model trained from the above training data can facilitate prediction of the surface-presenting peptides such that the prediction is performed in a manner that biases the selection towards peptides associated with a region in a space, in which the region being associated with outlier peptides in the training data set for which expression levels and peptide-presentation metrics were related in a manner that departed from the population-level relationship.

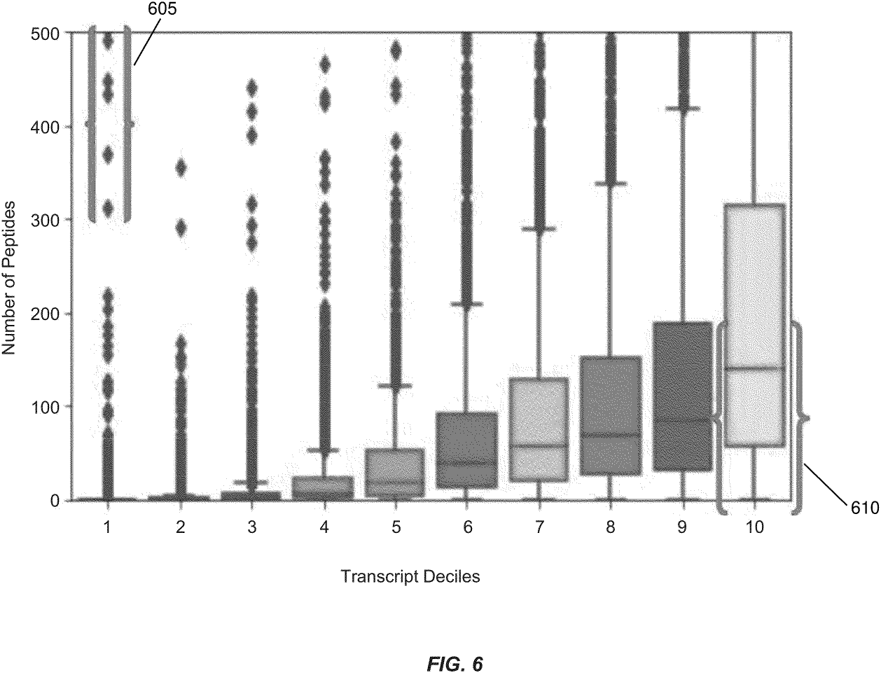

FIG. 6 shows a plot of comparison data between expected peptide counts based on gene expression levels and actual observed peptide counts, according to some embodiments. To generate the comparison data, all transcripts corresponding to HLA-binding peptides from the training data set can be identified and organized into a set of bins based on their respective gene expression levels. For example, as shown in FIG. 6, x-axis indicates 10 sections (e.g., a decile) in which the transcripts can be grouped based on their respective gene expression levels. The bars in each section indicates a measured amount of geneexpression levels that correspond to transcripts being grouped into the section. The y-axis of the plot shown in FIG. 6 specifies a number of peptides, and the diamond dots identify a quantity of peptides counted from cell lines of the training samples.

An initial hypothesis without the comparison data in FIG. 6 may indicate that an expected quantity of peptides is directly proportional to measured gene expression levels. With the comparison data in FIG. 6, however, one or more outliers that deviate from the initial hypothesis can be identified. A first outlier 605 includes a large quantity of peptides being observed in bin “1”, which indicates a very low amount of gene expression levels. A second outlier 610 includes almost no quantity of peptides being observed in bin “10”, which indicates a high amount of gene expression levels. As such, the comparison data in FIG. 6 indicates that measuring gene expression levels of HLA-binding peptides may not be sufficient enough to predict whether the HLA-binding peptides will actually be presented on a cell surface. The comparison data can be used to calculate a gene propensity score (“gps”) for a gene that encodes the HLA-binding peptides, at which the gene propensity score is predictive of whether a peptide will be presented on the cell surface. In some instances, the calculated gene propensity score is added as an additional feature of the training data set, such that the machine-learning model can be further trained based on the gene propensity score.

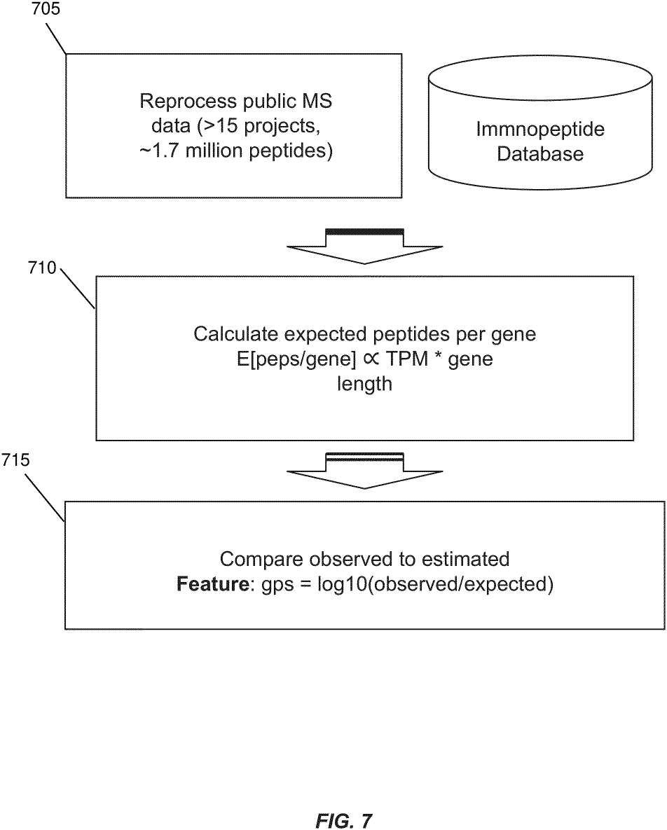

FIG. 7 shows a process for determining a gene propensity score used for training a machine-learning model, according to some embodiments. At block 705, immunopeptidomics data is obtained, which the immunopeptidomics data include expression levels of genes that encode HLA-binding peptides. For example, the immunopeptidomics data can be obtained by reprocessing existing mass spectrometry (MS) data or by accessing the immunopeptidomics data directly from a database (e.g., immunopeptide database).

At block 710, an expected number of peptides is calculated for a gene identified in the immunopeptidomics data. In particular, the expected number of peptides are calculated based on a number of transcripts (e.g., TPM) and a sequence length of the gene. At block 715, a ratio between the expected number of peptides and an observed number of peptides can be calculated to generate the gene propensity score (e.g., log10(observed/expected)). In some instances, the gene propensity score is added as an additional feature of the training data set.

### b) Comparison Between Expected Peptide Counts per Gene Region and Actual Observed Peptide Count

A second feature generated from the immunopeptidomics data can include comparison data between expected peptide counts based on expression levels within one or more regions in a given gene and actual observed peptide count corresponding to the one or more regions. In contrast to the first feature that identifies gene expression levels across various genes, the second feature identifies expression levels of regions within a single gene. An expected quantity of peptides can be generated based on the identified expression levels. The expected quantity can be compared with the observed quantity of peptides to identify the second feature for the training data set, in which the second feature is indicative of one or more surface-presentation characteristics of regions within the corresponding gene.

In some instances, the first feature and the second feature are combined into the training dataset. The trained machine-learning model trained from the training data with the combined features can further facilitate prediction of surface-presenting peptides prediction such that the prediction is biased towards peptides associated with scores predicting presentation to be more probable relative to a probability expected by a population-level relationship between expression and presentation of the peptides. In addition, the trained machine-learning model trained from the above training data can facilitate prediction of the surface-presenting peptides prediction such that the prediction is performed in a manner that biases the selection towards peptides associated with a region in a space, in which the region being associated with outlier peptides in the training data set for which expression levels and peptide-presentation metrics were related in a manner that departed from the population-level relationship.

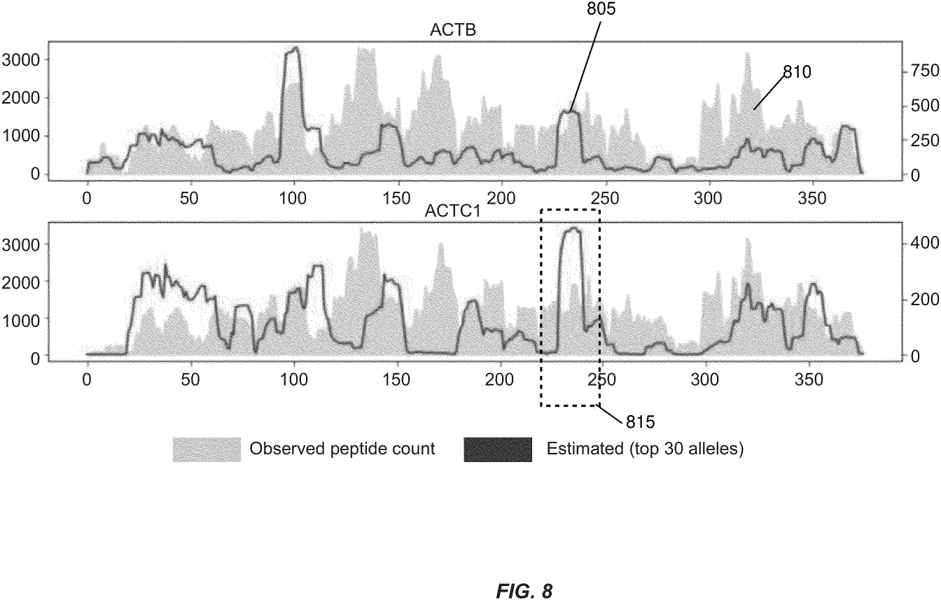

FIG. 8 shows a plot of comparison data between expected peptide counts for one or more regions within a gene and actual observed peptide count for the regions, according to some embodiments. For each genomic region of a given gene, gene expression levels can be calculated and the expected quantity of peptides can be measured. For example, the expected quantity of peptides for genomic regions of an ACTB gene are shown by a black plot line 805. The expected quantity can then be compared with an observed quantity of peptides per each genomic region, in which the observed quantity of peptides are shown by a gray area 810. Similar to the results in FIG. 6, several outliers can be identified within various genomic regions, in which the observed quantity of peptides are not proportional to the measured gene expression levels. For example, a region 815 of ACTC1 gene (e.g., region number 230) may indicate a very high expected quantity of peptides (e.g., > 3000 peptides), yet the observed quantity of the peptides in the same region 815 is actually much less than the expected quantity (e.g., approximately 1000 peptides). As such, the comparison data in FIG. 8 indicates that measuring region-level gene expression levels of HLA-binding peptides may not be sufficient enough to predict whether the HLA-binding peptides will actually be presented on a cell surface. The comparison data shown in FIG. 8 can be used to calculate a hotspot score (“hhs”) for a gene that encodes the HLA-binding peptides, at which the hotspot score is predictive of whether a peptide corresponding to a region of the gene will be presented on the cell surface.

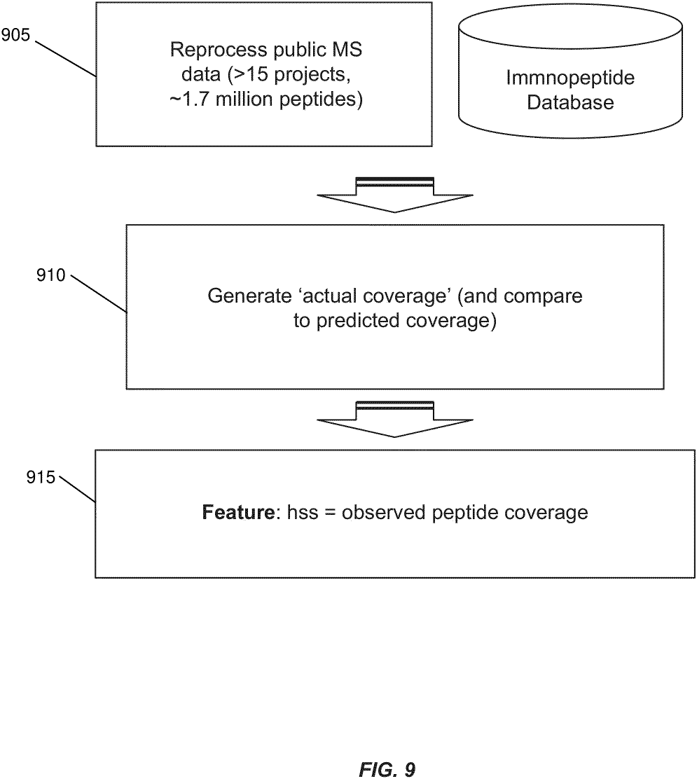

FIG. 9 shows a process for determining a hotspot score used for training a machine-learning model, according to some embodiments. At block 905, immunopeptidomics data is obtained, which the immunopeptidomics data include expression levels of genes that encode HLA-binding peptides. For example, the immunopeptidomics data can be obtained by reprocessing existing mass spectrometry (MS) data or by accessing the immunopeptidomics data directly from a database (e.g., immunopeptide database).

At step 910, predicted number of peptides for each region of a particular gene is compared to an actual number of peptides for the region. At step 915, a hotspot score is calculated for the particular gene, in which the hotspot score identifies a distribution of observed peptide counts across the regions of the gene (e.g., ACTB gene, ACTC1 gene).

### IV. Example Model Architecture For Predicting MHC-Binding Peptides Presented on a Cell Surface

The training data set can be used to train a machine-learning model for predicting surface-presenting peptides. The machine-learning model includes one or more sub-models configured to identify binding characteristics and surface-presentation characteristics of peptides in a sample. These sub-models can be separately trained with a corresponding subset of the training data set, such that each sub-model can predict the surface-presenting peptides based on parameters learned from features that correspond to the subsets.

### 1. Binding and Presentation Models

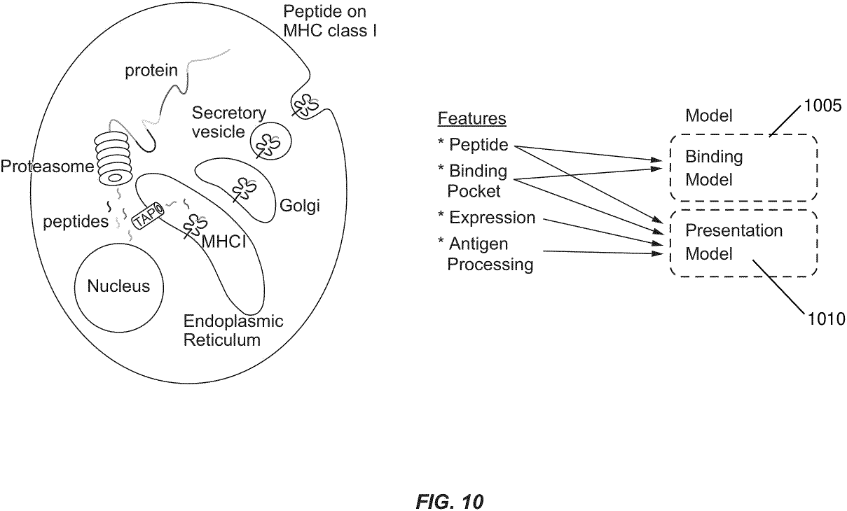

In some instances, the machine-learning model includes a binding model and a presentation model, each of which is trained to process different features of the input data. FIG. 10 shows an example of features used by a binding model 1005 and a presentation model 1010, according to some embodiments. The binding model 1005 can be trained using a training data set that includes information associated with a set of peptides (e.g., sequence of the MHC molecules that bind the peptides, peptide length). In some instances, the binding model 1005 includes one or more trained gradient-boosting algorithms. Gradient boosting refers to a machine-learning technique for regression and classification problems that produce a prediction model in the form of an ensemble of weak prediction models. The technique may build a model in a stage-wise fashion and generalizes the model by allowing optimization of an arbitrary differentiable loss function. Gradient boosting combines weak learners into a single strong learner in an iterative fashion. As each weak learner is added, a new model is fitted to provide a more accurate estimate of the response variable. The new weak learners can be maximally correlated with the negative gradient of the loss function, associated with the whole ensemble. Examples of the gradient boosting machines can include XGBoost and LightGBM. Additionally or alternatively, other types of machine-learning techniques can be used to build the binding model, including bagging procedures, boosting procedures, and/or random forest algorithms.

The presentation model 1010 can be trained not only using the information associated with peptides (e.g., peptide sequence, sequence of the MHC molecules that bind the peptides, peptide length), but also expression levels of the source protein from which the peptide was derived, surface-presentation characteristics of the peptides, gene propensity scores, and hotspot scores. The trained presentation model 1010 can thus identify binding characteristics of a given peptide and its surface-presentation characteristics, i.e., whether the peptide will be presented on a cell surface. Similar to the binding model 1005, the presentation model 1010 can include one or more trained gradient-boosting algorithms.

### 2. Model Architecture

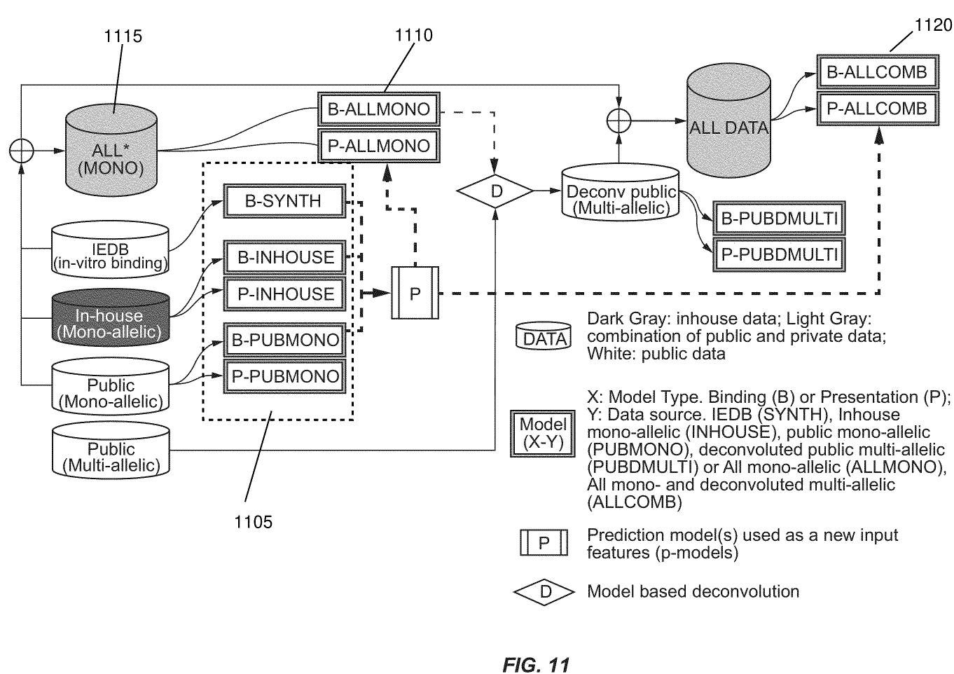

FIG. 11 illustrates an exemplary model architecture for training a machine-learning model for predicting surface-presenting peptides, according to some embodiments. As shown in FIG. 11, the training databases are shown in cylinders, in which the database includes various types of information including allelic data retrieved from publicly available sources. For example, the dark-gray cylinder includes immunopeptidomics data corresponding to genetically engineered mono-allelic cell lines (see FIG. 4). In another example, a training database may also include in-vitro binding data from publicly available data sources (e.g., an IEDB database represented by a white cylinder). In some instances, training data sets from each training database are used to separately train a corresponding binding model and a presentation model. In addition, the training databases can be combined into a larger training database to train its corresponding binding model and presentation model (e.g., “ALL (MONO)” light-gray cylinder of FIG. 11).

FIG. 11 additionally shows multiple sets of binding and presentation models that can be trained to predict the surface-presenting peptides. Each of the set of binding and presentation models are shown as being trained by different training data sets. In some instances, outputs generated from a first set of models 1105 (“initial models”) are used as input features for training a second set of models 1110 (“intermediate models”). For example, outputs generated by initial models that correspond to in-vitro binding, mono-allelic data from genetically engineered mono-allelic cell lines can be used as input features to train the intermediate models. The intermediate models can also be separately trained with training data derived from all mono-allelic data 1115. Further, outputs from the intermediate models can be deconvoluted and added into another training database that includes both mono-allelic and multi-allelic data. The outputs can be deconvoluted using one or more of the base mono-allelic base models, or an unsupervised clustering and alignment algorithm such as GibbsCluster.

The training data set from the database that includes the deconvoluted sets of peptides for each HLA allele can be used to train a third set of presentation and binding models 1120 (“final models”). The trained final models 1120 can be deployed to predict surface-presenting peptides. A purpose of building the training database to train the final models is to obtain as much allelic diversity as possible and avoid issues caused by underfilling or overfilling. Additionally or alternatively, trained intermediate models can also be deployed to predict the surface-presenting peptides, although performance levels of the trained final models tend to be superior than those of the trained intermediate models.

### V. Evaluating Performance Levels of the Machine-Learning Model

To evaluate performance of the trained machine-learning model, a test data set comprising several experimentally observed peptides that were not part of the training process and synthetic decoys was generated. The trained machine-learning model processed these candidate test peptides to output a score predictive of MHC Class I binding and cell-surface presentation, in which the machine-learning model was trained using large scale immunopeptidome training data sets as described above. The scores are then compared with a corresponding data derived from verified MHC-binding peptides that are presented on a cell surface to identify performance levels of the trained machine-learning model. The output scores were also benchmarked against NetMHCpan 4.0 (an existing platform for predicting binding of peptides to MHC molecules), and the trained machine-learning algorithm demonstrated higher overall sensitivity and specificity. Based on the output scores, an antigen burden score of the predicted peptides can be calculated using peptides having the output scores that pass a confidence threshold.

In another example, the trained machine-learning model was tested and evaluated using experimentally generated peptides, using mass spectrometry-based immunopeptidomics approaches described above, from tissue samples, mixed with decoys in a 1:999 ratio. The positive predictive value in the top 0.1% predicted ligands by the trained machine-learning model is significantly higher compared to NetMHCPan 4.0, which is a publicly available tool regarded as the gold standard for prediction of MHC-binding peptides. In yet another example, the trained machine-learning model was also evaluated using a leave-one-out analysis, in which a high-degree of agreement was observed between motifs in raw data and motifs predicted by trained machine-learning model.

### 1. Model Evaluation on Mono-Allelic Data

### a) Positive Predictive Value

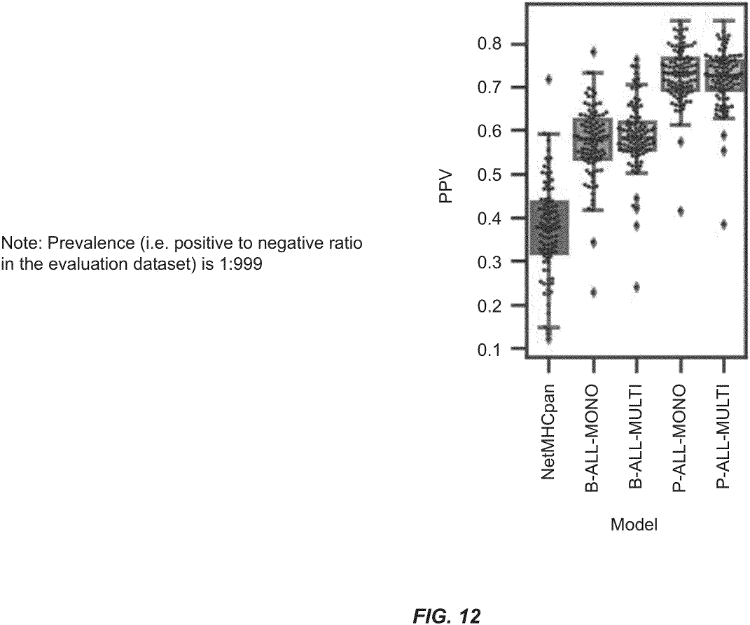

FIG. 12 shows performance levels of trained binding models and trained presentation models based on 10% held-out data and measured in terms of positive predictive value, according to some embodiments. The evaluation data is based on mono-allelic immunopeptidomics data. The positive predictive value (PPV) is defined as a proportion of predicted positives of the trained machine-learning model which are actual positives. Thus, PPV reflects a probability a predicted positive is a true positive. In the evaluation dataset, the prevalence identifying positive to negative ratio is 1:999.

As shown in FIG. 12, the median PPV value corresponding to NetMHCpan is approximately 0.4. In comparison, the trained binding models perform relatively better than NetMHCpan, in which the binding model trained with mono-allelic data having a median PPV value of approximately 0.6 and the binding model trained with mono- and multi-allelic data having a comparable median PPV value of approximately 0.6. The first trained presentation model trained with mono-allelic data and the second trained presentation model with mono- and multi-allelic data perform significantly better than NetMHCpan, having median PPV values of approximately 0.7. A 0.1 PPV-value difference of performance can be attributed to the evaluation data being derived from mono-allelic data.

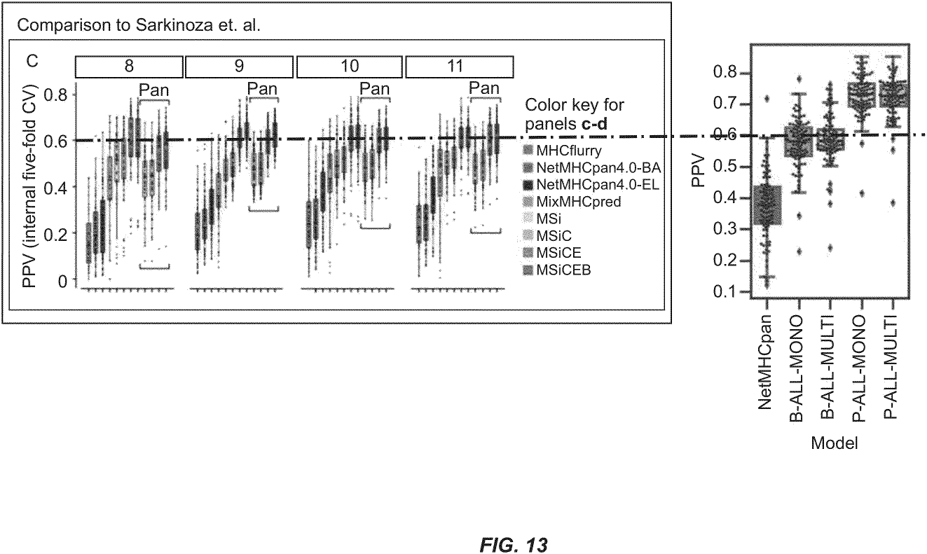

FIG. 13 shows a comparison of performance levels of the trained machine-learning model compared to conventional techniques for predicting MHC-binding peptides. As shown in FIG. 13, other conventional techniques show median PPV values at approximately 0.6, which are equivalent to the median PPV values corresponding to the trained binding models. Compared to the above models, the trained presentation models tend to perform better having median PPV values of approximately 0.7.

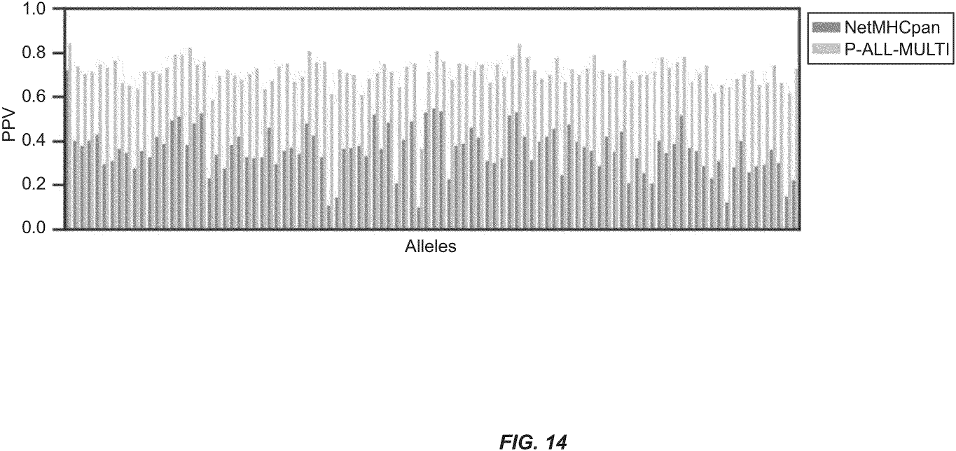

FIG. 14 shows a comparison of performance levels of a trained presentation model across various alleles, compared to conventional techniques for predicting MHC-binding peptides. The PPV values for each allele are shown for NetMHCpan and the trained presentation model. As shown in FIG. 14, the PPV values corresponding to the trained presentation model are significantly higher than those of the NetMHCpan across every single allele. As such, the trained presentation model has demonstrated a significant improvement over NetMHCpan for predicting MHC-binding peptides that are presented and expressed on a cell surface.

### b) Leave-One Out Analysis

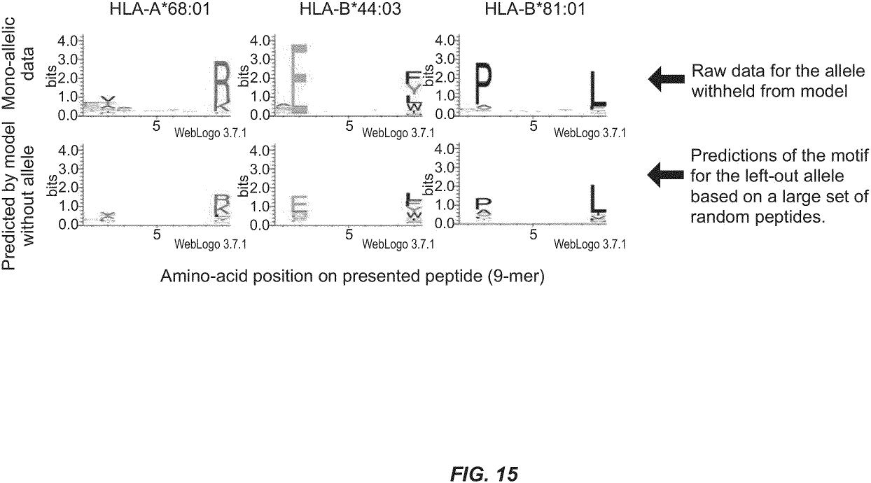

FIG. 15 shows results from a leave-one out analysis of a trained presentation model, according to some embodiments. The leave-one out analysis can be used to evaluate whether the trained presentation model can predict surface-presented peptides corresponding to alleles that are absent from any training data, in order to demonstrate performance of the trained presentation model in discovering unknown types of MHC-binding peptides that will likely be presented on a cell surface. To implement the leave-one out analysis, the presentation model was trained with a training data set that excludes training data corresponding to one specific allele. After the training, the trained machine-learning model was evaluated by processing 500,000 random peptides to predict surface-presenting peptides, in which at least some of the MHC-binding peptides are encoded by the excluded allele. Motifs of the predicted MHC-binding peptides were compared with those derived from raw data in which the specific allele was available, in order to assess accuracy of the prediction of the peptide by the trained machine-learning model.

As shown in FIG. 15, the motifs corresponding to the predicted surface-presenting peptides substantially match the motifs of the peptide corresponding to the excluded allele, in which the motifs show comparable amino-acid expression levels across 9 positions of the subject peptide. For example, the number two position of the peptide corresponding to HLA-B*44.03 shows high expression levels of Glutamic acid (“E”) in raw data. The predicted MHC-binding peptides presented on the cell surface also show high expression levels of Glutamic acid at the same number two position. Thus, the trained machine-learning model can accurately predict MHC-binding peptides presented on the cell surface even when corresponding alleles are not part of the training data.

### c) Precision and Recall

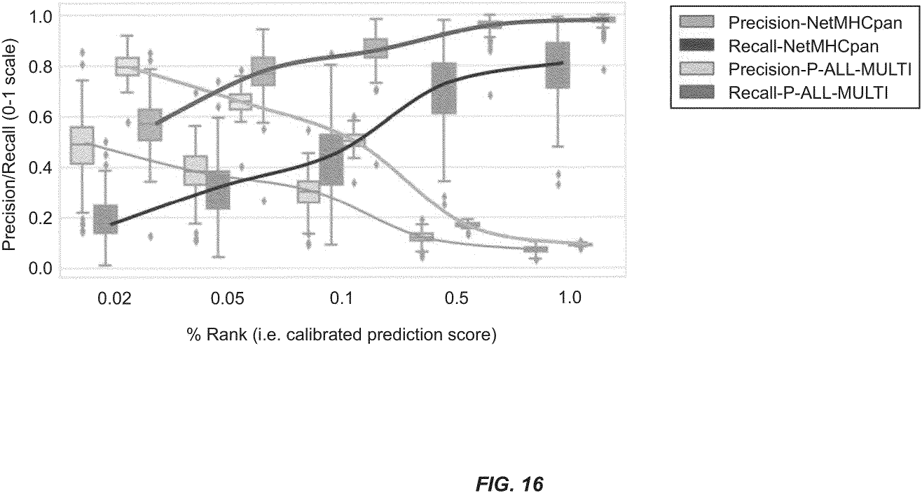

FIG. 16 shows a graph depicting precision and recall values for evaluating a trained machine-learning model, according to some embodiments. Precision-Recall can be a useful measure of success of prediction. In information retrieval, precision is a measure of result relevancy, while recall is a measure of how many truly relevant results are returned. A high precision relates to a low false positive rate, and high recall relates to a low false negative rate. High scores for both precision and recall can demonstrate that a given classifier is returning accurate results (high precision), as well as returning a majority of all positive results (high recall). The performance of the trained machine-learning model was evaluated based on held-out mono allelic data using 10% of immunopeptidomics data from training mixed with synthetic negative examples in a 1:999 ratio. The x-axis of the graph corresponds to a set of rank-percentile thresholds ranging from 0.02 to 1.0, which would identify surface-presenting peptides that are within the specified rank-percentile threshold to be considered for either binding or presentation.

As shown in FIG. 16, the trained machine-learning model corresponds to high precision over all recall values compared to NetMHCpan. The difference is further highlighted among top 1% peptides in the test data, in which the median precision over recall is approximately 0.8 / 0.6 for the trained machine-learning model whereas the median precision over recall is approximately 0.5 / 0.2 for NetMHCpan. Thus, the trained machine-learning model can demonstrate an improvement of predicting the surface-presenting peptides over NetMHCpan, which returned accurate results, as well as returning a majority of all positive results.

### 2. Model Evaluation on Multi-Allelic (Tissue) Samples

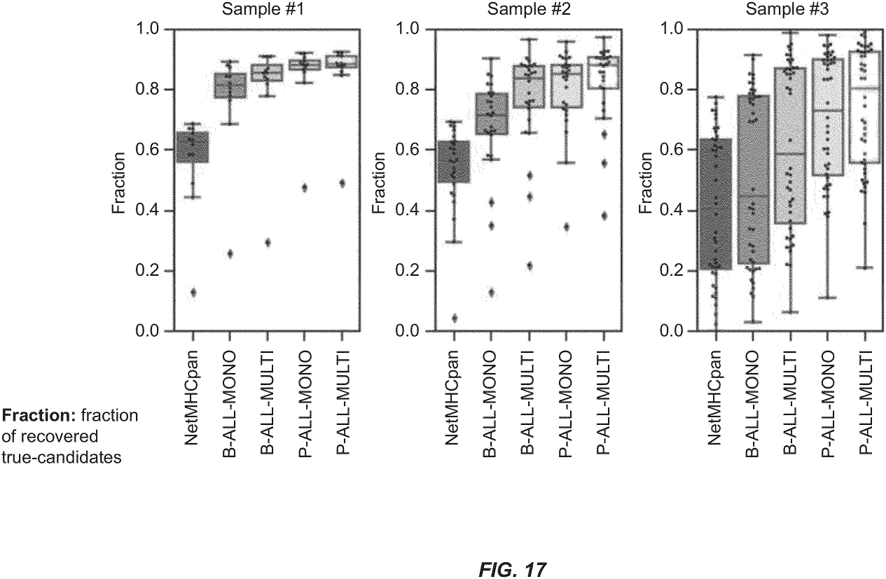

The performance levels of the trained machine-learning model using multi-allelic samples further demonstrate an improvement of predicting surface-presenting peptides over conventional techniques such as NetMHCpan. FIG. 17 shows a box plot that indicates performance levels of the trained machine learning models across different tissue samples, according to some embodiments. In FIG. 17, three types of tissue samples were processed by the trained machine-learning model to generate a fraction of recovered true-candidates that correspond to the surface-presenting peptides. A higher fraction can thus indicate that the trained machine-learning model can demonstrate a high performance level in accurately identifying the surface-presenting peptides across different tissue samples.

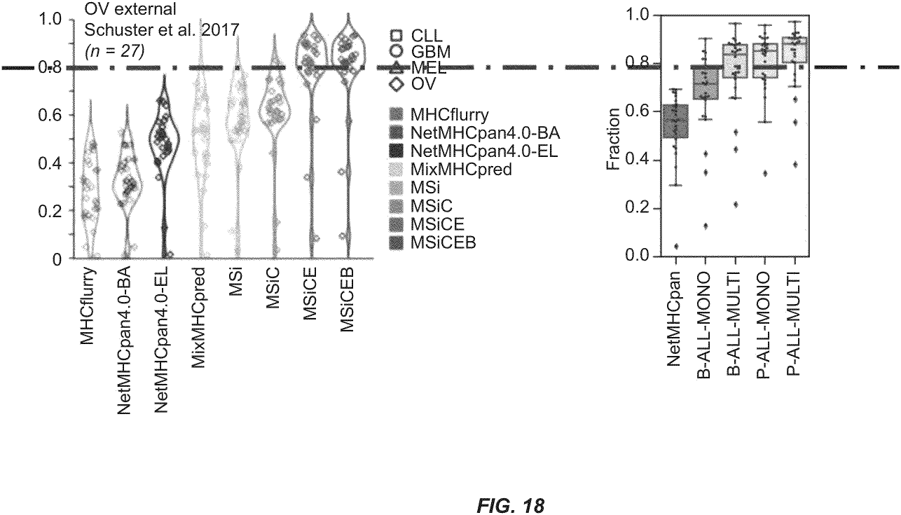

For example, the fraction value corresponding to NetMHCpan is approximately 0.65. This fraction value indicates that NetMHCpan was able to predict approximately 65% of the surface-presenting peptides that are actually present in the tissue sample. In comparison, the trained binding models perform better than NetMHCpan, in which the binding model trained with mono-allelic data having a fraction value of approximately 0.81 and the binding model trained with mono- and multi-allelic data having a fraction value of approximately 0.85. The first trained presentation model trained with mono-allelic data and the second trained presentation model with mono- and multi-allelic data perform even better, both corresponding to fraction values of approximately 0.9. Thus, the trained presentation models identified approximately 90% of the surface-presenting peptides experimentally identified in the tissue samples. Similar improvements of prediction of surface-presenting peptides were shown in other tissue samples. FIG. 18 shows a graph that compares performance levels of trained machine learning models and other conventional techniques, according to some embodiments.

### VI. Example Process for Predicting MHC-Binding Peptides Presented on a Cell Surface

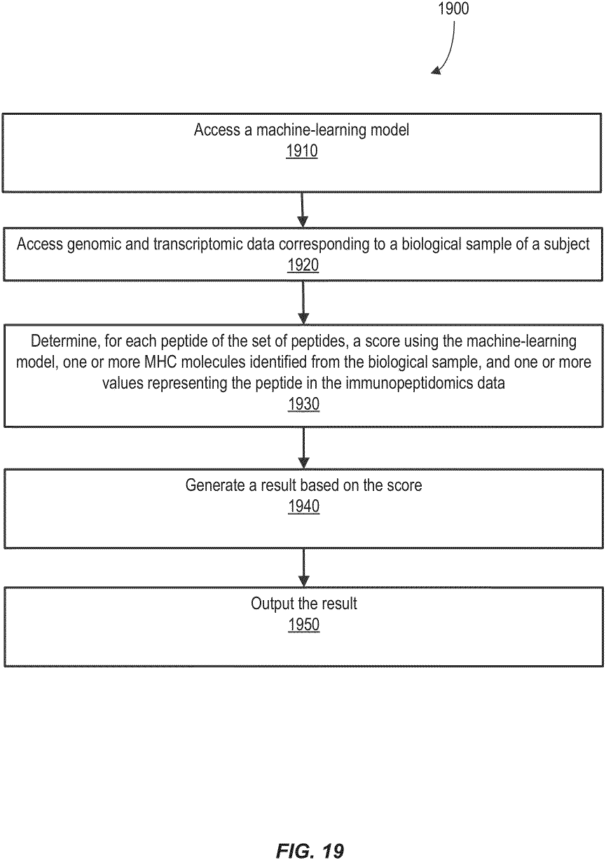

FIG. 19 includes a flowchart 1900 illustrating an example of a method of predicting surface-presenting peptides, according to certain embodiments. Operations described in flowchart 1900 may be performed by, for example, a computer system implementing a trained machine-learning model, such as a trained binding and presentation models. Although flowchart 1900 may describe the operations as a sequential process, in various embodiments, many of the operations may be performed in parallel or concurrently. In addition, the order of the operations may be rearranged. An operation may have additional steps not shown in the figure. Furthermore, embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At operation 1910, a computer system accesses a machine-learning model. The machine-learning model was trained using a training data set that included, for each peptide of a plurality of peptides identified by the training data set: protein characteristics of an MHC molecule (e.g., an HLA allele) that binds and presents the peptide on a cell surface; and one or more expression levels representing an expression level of a gene encoding the peptide; and one or more peptide-presentation metrics representing a quantity of peptides detected as having been presented by the MHC molecule. The machine-learning model is configured to generate an output that indicates an extent to which the one or more expression levels and the one or more peptide-presentation metrics are related in accordance with a population-level relationship between expression and presentation.

At operation 1920, the computer system accesses genomic and transcriptomic data corresponding to a biological sample of a subject. The genomic data and transcriptomic data of the biological sample processed to identify candidate neoantigens (peptides). The genomic and transcriptomic data identifies one or more MHC molecules from the biological sample and includes, for each peptide of a set of peptides identified from the tissue sample (e.g., candidate neoantigens), one or more values representing the peptide. At least one of the one or more values can be determined based on processing of the tissue sample. The one or more values can correspond to a type of the peptide, length of the peptide, an allele that binds the peptide, and expression of a gene region that encodes the peptide.

At operation 1930, the computer system determines, for each peptide of the set of peptides, a score using the machine-learning model, the one or more MHC molecules identified from the biological sample, and the one or more values representing the peptide in the genomic and transcriptomic data. In some instances, the computer system uses the trained machine-learning model processes the one or more values to output, for a given peptide, a score predictive of MHC-molecule binding and presentation.

At operation 1940, the computer system generates a result based on the score. The results may include an incomplete subset of peptides that exceeds a predefined threshold, in which the subset of peptides are predicted to be surface-presenting peptides. In some instances, the result may include motifs that correspond to each of the subset of peptides. Additionally or alternatively, the results can include a subset of peptides having scores above a particular ranking percentile of scores (e.g., 0.02). In some instances, the result indicates, for each peptide of the set of peptides, whether the peptide is a surface-presenting peptide, i.e., a peptide that binds to a corresponding MHC molecule and is presented on a cell surface.

In some instances, the computer system selects an incomplete subset of the set of peptides, in which an identification of the incomplete subset is performed in a manner that biases the selection towards peptides associated with a region in a space, the region being associated with outlier peptides in the training data set for which expression levels and peptide-presentation metrics were related in a manner that departed from the population-level relationship.

At operation 1950, the computer system outputs the result. Process 1900 terminates thereafter.

### VII. Computing Environment

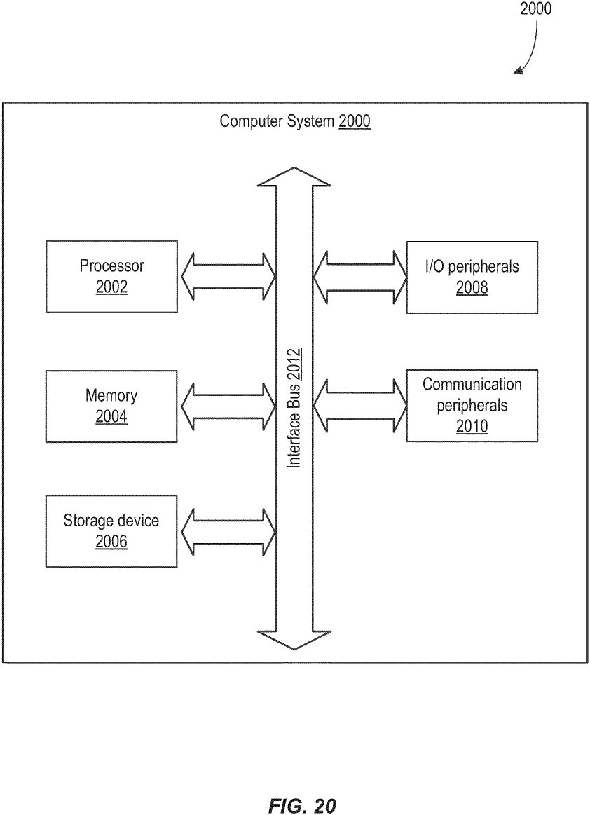

FIG. 20 illustrates an example of a computer system 2000 for implementing some of the embodiments disclosed herein. Computer system 2000 may have a distributed architecture, where some of the components (e.g., memory and processor) are part of an end user device and some other similar components (e.g., memory and processor) are part of a computer server. Computer system 2000 includes at least a processor 2002, a memory 2004, a storage device 2006, input/output (I/O) peripherals 2008, communication peripherals 2010, and an interface bus 2012. Interface bus 2012 is configured to communicate, transmit, and transfer data, controls, and commands among the various components of computer system 2000. Processor 2002 may include one or more processing units, such as CPUs, GPUs, TPUs, systolic arrays, or SIMD processors. Memory 2004 and storage device 2006 include computer-readable storage media, such as RAM, ROM, electrically erasable programmable read-only memory (EEPROM), hard drives, CD-ROMs, optical storage devices, magnetic storage devices, electronic non-volatile computer storage, for example, Flash® memory, and other tangible storage media. Any of such computer-readable storage media can be configured to store instructions or program codes embodying aspects of the disclosure. Memory 2004 and storage device 2006 also include computer-readable signal media. A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with computer system 2000.

Further, memory 2004 includes an operating system, programs, and applications. Processor 2002 is configured to execute the stored instructions and includes, for example, a logical processing unit, a microprocessor, a digital signal processor, and other processors. Memory 2004 and/or processor 2002 can be virtualized and can be hosted within another computing system of, for example, a cloud network or a data center. I/O peripherals 2008 include user interfaces, such as a keyboard, screen (e.g., a touch screen), microphone, speaker, other input/output devices, and computing components, such as graphical processing units, serial ports, parallel ports, universal serial buses, and other input/output peripherals. I/O peripherals 2008 are connected to processor 2002 through any of the ports coupled to interface bus 2012. Communication peripherals 2010 are configured to facilitate communication between computer system 2000 and other computing devices over a communications network and include, for example, a network interface controller, modem, wireless and wired interface cards, antenna, and other communication peripherals.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of the methods disclosed herein may be performed in the operation of such computing devices. The order of the blocks presented in the examples above can be varied-for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A method comprising:
accessing a machine-learning model, wherein the machine-learning model:
was trained using a training data set that included, for each peptide of a plurality of peptides identified by the training data set:
protein characteristics of a major histocompatibility complex (MHC) molecule that binds and presents the peptide;
one or more expression levels representing an expression level of a gene encoding the peptide; and
one or more peptide-presentation metrics representing a quantity of peptides detected as having been presented by the MHC molecule;
is configured to generate an output that indicates an extent to which the one or more expression levels and the one or more peptide-presentation metrics are related in accordance with a population-level relationship between expression and presentation;
accessing genomic and transcriptomic data corresponding to a biological sample of a subject, wherein the genomic and transcriptomic data identifies one or more MHC molecules from the biological sample and includes, for each peptide of a set of peptides identified from the tissue sample, one or more values representing the peptide, at least one of the one or more values having been determined based on processing of the tissue sample;
determining, for each peptide of the set of peptides, a score using the machine-learning model, the one or more MHC molecules identified from the biological sample, and the one or more values representing the peptide;
generating a result based on the scores; and
outputting the result.

2. The method of claim 1, further comprising:
selecting an incomplete subset of the set of peptides based on the scores, wherein an identification of the incomplete subset is performed in a manner that biases the selection towards peptides associated with scores predicting presentation to be more probable relative to a probability expected by the population-level relationship, wherein the result includes the incomplete subset of the set of peptides.

3. The method of claim 1, further comprising:
selecting an incomplete subset of the set of peptides based on the scores, wherein an identification of the incomplete subset is performed in a manner that biases the selection towards peptides associated with a region in a space, the region being associated with outlier peptides in the training data set for which expression levels and peptide-presentation metrics were related in a manner that departed from the population-level relationship.

4. The method of claim 1, wherein the result includes, for each peptide of one or more of the set of peptides, an identification of the peptide and the score.

5. The method of claim 1, wherein, for each peptide in the set of peptides, the one or more values representing the peptide are generated based on an amino-acid sequence of the peptide, an indication of whether the peptide binds to one or more binding pockets of the MHC molecule, an expression level of the peptide in the tissue sample, and/or a length of the peptide.

6. The method of claim 1, wherein the training data set is derived from mono-allelic data corresponding to peptides derived from mono-allelic cell lines and/or multi-allelic data corresponding to peptides derived from other tissue samples.

7. The method of claim 1, wherein the score corresponding to a peptide of the set of peptides corresponds to a predicted probability as to whether the peptide will bind to the MHC molecule and be presented on a cell surface.

8. The method of claim 1, wherein the machine-learning model includes one or more trained gradient boosting algorithms.

9. The method of claim 1, wherein the machine-learning model includes a first sub-model trained with a first subset of the training data set that includes, for each peptide of the plurality of peptides, a sequence corresponding to the peptide, a sequence of an MHC molecule that binds the peptide, and/or a length of peptides.

10. The method of claim 9, wherein the machine-learning model includes a second sub-model trained with a second subset of the training data set that includes, for each peptide of the plurality of peptides, one or more expression levels of a source protein from which the peptide was derived and surface-presentation characteristics of the peptide.

11. The method of claim 10, wherein each of the first and second sub-models was trained based on one or more outputs generated by another set of sub-models.

12. A method comprising:
accessing a composite machine-learning model comprising: (i) a first machine-learning model configured to predict whether a peptide from a biological sample will bind to at least one major histocompatibility complex (MHC) molecule; and (ii) a second machine-learning model configured to predict whether the peptide from the biological sample will be presented on a cell surface, wherein:
the first machine-learning model is trained using a first training data set that includes a first set of input features, wherein each of the first set of input features includes one or more binding characteristics of a peptide and a corresponding MHC molecule that binds the peptide, and wherein the first set of input features are determined by processing one or more mono-allelic cell lines; and
the second machine-learning model is trained using a second training data set that includes a second set of input features, wherein each of the second set of input features includes one or more surface-presenting characteristics of the peptide and the corresponding MHC molecule, and wherein each of the second set of input features are determined by deconvoluting data from one or more mono-allelic cell lines and one or more multi-allelic tissue samples using the first machine-learning model; and
availing the composite machine-learning model, wherein the composite machine-learning model is configured to predict, from a set of peptides, an incomplete subset of peptides that will bind to the at least one MHC molecule and be presented on the cell surface.

13. A system comprising:
one or more data processors; and
a non-transitory computer readable storage medium containing instructions which, when executed on the one or more data processors, cause the one or more data processors to perform operations comprising:
accessing a machine-learning model, wherein the machine-learning model:
was trained using a training data set that included, for each peptide of a plurality of peptides identified by the training data set:
protein characteristics of a major histocompatibility complex (MHC) molecule that binds and presents the peptide;
one or more expression levels representing an expression level of a gene encoding the peptide; and
one or more peptide-presentation metrics representing a quantity of peptides detected as having been presented by the MHC molecule;
is configured to generate an output that indicates an extent to which the one or more expression levels and the one or more peptide-presentation metrics are related in accordance with a population-level relationship between expression and presentation;
accessing genomic and transcriptomic data corresponding to a biological sample of a subject, wherein the genomic and transcriptomic data identifies one or more MHC molecules from the biological sample and includes, for each peptide of a set of peptides identified from the tissue sample, one or more values representing the peptide, at least one of the one or more values having been determined based on processing of the tissue sample;
determining, for each peptide of the set of peptides, a score using the machine-learning model, the one or more MHC molecules identified from the biological sample, and the one or more values representing the peptide;
generating a result based on the scores; and
outputting the result.

14. The system of claim 13, wherein the instructions further cause the one or more data processors to perform operations comprising:
selecting an incomplete subset of the set of peptides based on the scores, wherein an identification of the incomplete subset is performed in a manner that biases the selection towards peptides associated with scores predicting presentation to be more probable relative to a probability expected by the population-level relationship, wherein the result includes the incomplete subset of the set of peptides.

15. The system of claim 13, wherein the result includes, for each peptide of one or more of the set of peptides, an identification of the peptide and the score.

16. The system of claim 13, wherein, for each peptide in the set of peptides, the one or more values representing the peptide are generated based on an amino-acid sequence of the peptide, an indication of whether the peptide binds to one or more binding pockets of the MHC molecule, an expression level of the peptide in the tissue sample, and/or a length of the peptide.

17. The system of claim 13, wherein the training data set is derived from mono-allelic data corresponding to peptides derived from mono-allelic cell lines and/or multi-allelic data corresponding to peptides derived from other tissue samples.

18. The system of claim 13, wherein the score corresponding to a peptide of the set of peptides corresponds to a predicted probability as to whether the peptide will bind to the MHC molecule and be presented on a cell surface.

19. The system of claim 13, wherein the machine-learning model includes one or more trained gradient boosting algorithms.

20. The system of claim 13, wherein the machine-learning model includes a first sub-model trained with a first subset of the training data set that includes, for each peptide of the plurality of peptides, a sequence corresponding to the peptide, a sequence of an MHC molecule that binds the peptide, and/or a length of peptides.
